(19) United States
(12) Patent Application Publication
(10) Pub. No.: US 2023/0330284 A1
(43) Pub. Date: Oct. 19, 2023

(54) DISINFECTING DEVICE INCLUDING ROTATING CHAMBER BASE

(71) Applicant: Cleanbox Technology, Inc., Nashville, TN (US)

(72) Inventor: David Georgeson, Vista, CA (US)

(21) Appl. No.: 18/025,678

(22) PCT Filed: Sep. 10, 2021
(86) PCT No.: PCT/US2021/049745

§ 371 Date: Mar. 10, 2023

Related U.S. Application Data
(60) Provisional application No. 63/076,572, filed on Sep. 10, 2020.

(51) Int. Cl. A61L 2/10

(52) U.S. Cl. CPC A61L 2/10; A61L 2202/11; A61L 2202/122; A61L 2202/121

(57) ABSTRACT

The present disclosure provides devices including a rotating chamber and methods of disinfecting (e.g., sanitizing or decontaminating) an object using same.

### PRIORITY CLAIM

This application claims priority to U.S. Provisional Patent Application Ser. No. 63/076,572, filed Sep. 10, 2020, the entire contents of which are incorporated herein by reference and relied upon.

### FIELD

The present disclosure provides devices including a rotating chamber and methods of disinfecting (e.g., sanitizing or decontaminating) an object using same.

### BACKGROUND

Devices that claim to disinfect surfaces of objects using only UV light radiation often fail to perform as advertised. More specifically, existing commercial devices typically require long processing times, rearrangement of the objects during processing, and/or employ UV lamps that destroy common materials. Many of such devices use internal racks that are opaque to UV radiation, compounding these performance problems.

A need persists for devices that efficiently and effectively disinfect the surfaces of a variety of objects without degrading the objects themselves. The present disclosure satisfies this need.

### SUMMARY

One aspect of the disclosure provides a device configured to disinfect an object including a surface, the device including a lid with at least one UVC light radiation source, a chamber configured to house the object. The chamber includes at least one side wall including at least one UVC light radiation source and a rotatable base comprising a material translucent or transparent to UVC light radiation, such as quartz glass, and at least one UVC light radiation source disposed below the rotatable base.

Other aspects of the disclosure provide a device as above, wherein the at least one side wall includes an inner surface comprising a material that reflects at least about 85% of UVC light radiation, such as polytetrafluoroethylene (“PTFE”). The inner surface may reflect at least about 90% of light radiation having a wavelength of 265 nm. The lid may include an inner surface comprising a material that reflects at least about 85% of UVC light radiation, such as PTFE. The lid inner surface may reflect at least about 90% of light radiation having a wavelength of 265 nm. The rotatable base may be configured to rotate relative to the UVC light radiation sources. The enclosure and fan may be configured to force air around but not into the chamber 510. The UVC light radiation sources may be UVC LED lamps. Each UVC LED lamp may be a 50-70 mW UVC LED lamp. Each UVC LED lamp may be configured to emit UVC light radiation in a conical shape having a cone angle α of about 90°. The rotatable base 540 may be configured to rotate at a rate of about 5 rpm to about 20 revolutions per minute (“rpm”). The rotatable base may be configured to rotate at a rate of about 10 rpm.

In yet other aspects of the disclosure, the device may be configured to kill at least 99.9% (e.g., log-3), at least 99.99% (e.g., log-4), at least 99.999% (e.g., log-5), or at least 99.9999% (e.g., log-6) of pathogens on the surface of the object. The light radiation sources are configured such that pathogens are killed within about 5 minutes of an initial contact with UVC light radiation from the UVC light radiation sources. The light radiation sources may be configured such that pathogens are killed within about 3 minutes of an initial contact with UVC light radiation from the UVC light radiation sources. The light radiation sources are configured such that pathogens may be killed within about 1 minute of an initial contact with UVC light radiation from the UVC light radiation sources.

In further aspects of the disclosure a chamber is provided comprising UVC light radiation emitted at an average intensity of about 0.5 mW/cm² to about 0.6 mW/cm² at 265 nm. The intensity of the UVC light radiation may be not less than about 0.4 mW/cm² at 265 nm at any point within the chamber. The chamber may be at least partially enclosed by a polytetrafluoroethylene (“PTFE”) surface. The chamber may include a base comprising, consisting essentially of, or consisting of quartz glass. The chamber may be enclosed by an enclosure consisting essentially of PTFE and quartz glass. The UVC light radiation may be supplied by one or more UVC LED lamps. The base may be rotated relative to a source of the UVC light radiation. The chamber may have a volume of about 200 in³ (about 3.28 L) to about 500 in³ (about 8.19 L), or in another embodiment about 314 in³ (about 5.14 L). The UVC light radiation may be supplied by about 25 to about 40 UVC LED lamps. The UVC light radiation may be supplied by not more than 32 UVC LED lamps. The chamber may include a plurality of UVC LED lamps disposed on a lid of the chamber, a plurality of the UVC LED lamps are disposed below a base of the chamber, and a plurality of the UVC LED lamps are disposed about one or more side walls of the chamber.

### BRIEF DESCRIPTION OF THE FIGURES

The present invention will be described in even greater detail below based on the exemplary figures. The invention is not limited to the exemplary embodiments. All features described and/or illustrated herein can be used alone or combined in different combinations in embodiments of the invention. The features and advantages of various embodiments of the present invention will become apparent by reading the following detailed description with reference to the attached drawings which illustrate the following:

FIG. 1 is a perspective view of a device for disinfecting (e.g., sanitizing) the surface of an object consistent with one embodiment of the present disclosure.

FIG. 2 is a perspective view of the device of FIG. 1.

FIG. 3 is a perspective view of the device of FIGS. 1-2 with the side walls and bottom wall removed.

FIG. 4 is a perspective view of a chamber and rotatable base of the device of FIGS. 1-3.

FIG. 5 is a perspective view of the rotatable base of the device of FIGS. 1-4.

FIG. 6 is a perspective view of one embodiment of a motor-driven rotatable base suitable for use with a device consistent with FIGS. 1-5.

FIG. 7 is a perspective view of a fan and vents disposed below the rotatable base of the device of FIGS. 1-6.

FIG. 8 is a representative view of UVC light radiation patterns produced by a plurality of UVC LED lamps arranged in one configuration suitable for use in a device consistent with FIGS. 1-7.

### DETAILED DESCRIPTION

For purposes of the description hereinafter, the terms “upper, lower, right, left, vertical, horizontal, top, bottom, lateral, longitudinal” and other terms of orientation or position and derivatives thereof, shall relate to the invention as it is depicted in the figures. The term “configured” or “configuration” will be understood as referring to a structural size and/or shape. It is to be understood that the invention may assume alternative variations and step sequences, except where expressly specified to the contrary. It is also to be understood that the specific systems and processes illustrated in the attached drawings, and described in the following specification, are simply exemplary examples of the invention. Hence, specific dimensions and other physical characteristics related to the examples disclosed herein are not to be considered as limiting.

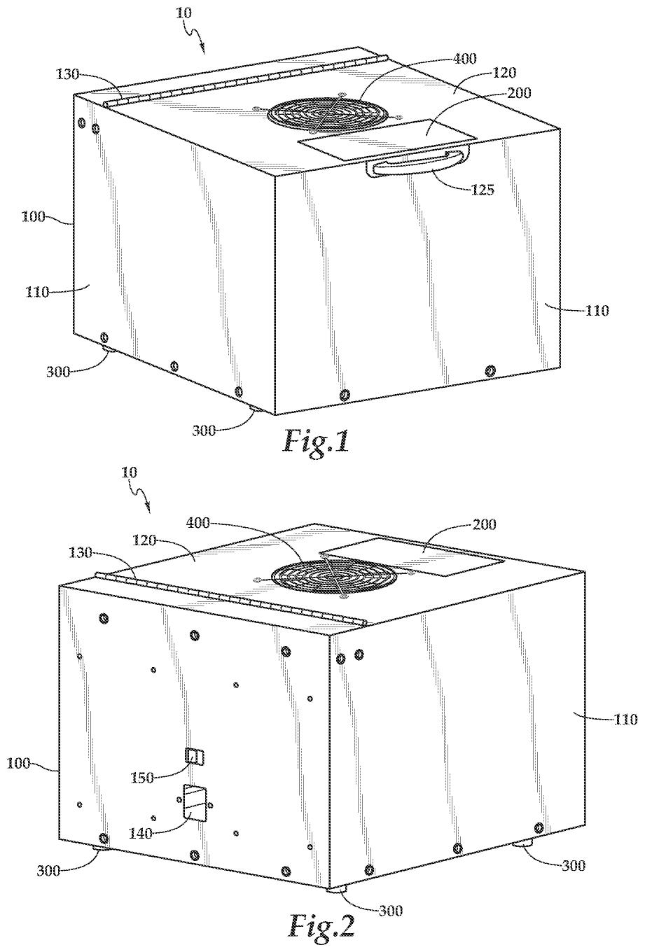

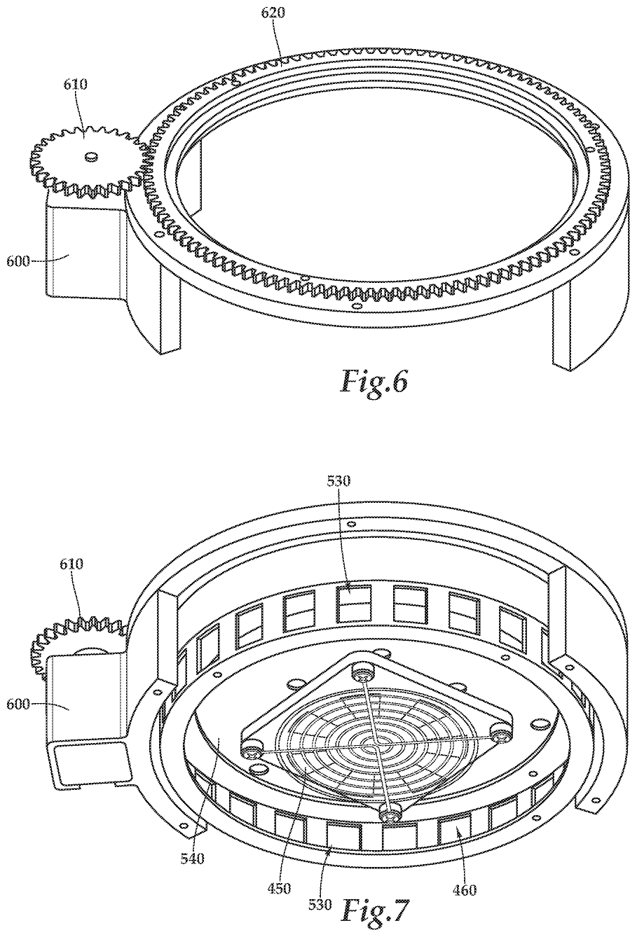

Referring generally to FIGS. 1-8, the present disclosure provides devices including a rotatable chamber and methods of disinfecting (e.g., sanitizing) the surface of an object using same. Turning to FIGS. 1 and 2, a device 10 is shown that incorporates the elements of the disinfecting chamber of the disclosure, exterior elements being shown.

In particular, the device 10 includes an enclosure 100. The enclosure 100 may be any suitable size and shape taking into consideration that exposing objects in the enclosure with a selected intensity of UVC light radiation is a feature of the invention, which is affected by and is a function of the volume of the enclosure and the ability to provide the volume with UVC light radiation in a sufficient density or amount. The enclosure 100 may have an overall shape of square or rectangular or any suitable shape configured to accommodate objects to be treated inside. The enclosure 100 may be made of metal, or plastic, or composite materials, or any suitable material, preferably materials and surfaces that are (a) opaque to UVC radiation, and (b) not porous and enable cleaning, and (c) do not, to the extent possible, harbor bacteria, virus, or other pathogens, or contaminants.

The enclosure 100 includes one or more sidewalls 110, for example four sidewalls if the enclosure is rectangular. The enclosure 100 is closed with a lid 120 that is sized to accommodate the passage of objects to be treated within the enclosure and may be made of the same or different material as the sidewalls 110. The lid 120 includes a handle 125 or any suitable means of operating the lid, e.g., for opening, closing or otherwise manipulating the position of the lid. The lid 120 is attached via a hinge 130, which may be a piano-type hinge or any suitable hinge type. The lid 100 preferably forms a seal or has a fit so as to prevent light and air from escaping the interior of the enclosure and therefore has a function directed to preventing contaminants contained within the enclosure from escaping the enclosure during treatment.

A fan 400 may be disposed in the lid 120 for cooling and/or venting purposes. A preferred embodiment is configured such that the fan pulls air out of the interior of the enclosure 100 to cool heat-producing elements of the device 10 but the fan is not fluidly connected to an interior portion 510 of the enclosure 100 that contains objects to be treated as will be explained below. In this manner, the device 10 does not spread pathogens and/or contaminants contained within the device 10 to the outside of the device 10.

A control panel or control input device 200 may be disposed in or on the lid 120. The control panel 200 may be in the form of a touch screen panel, which is easy to keep clean and simple to operate. The control panel 200 may be provided with touchable icons (not shown) as is well known to select and start/stop various operations of the device 10 and adjust operating parameters thereof.

Optionally, the enclosure 100 includes one or more foot 300 on a bottom surface there that is for supporting the enclosure on a surface, like a work surface, and may be non-skid and/or non-marring to keep the enclosure from moving or marring the surface on which it is being used. The foot 300 may be a plurality of circular or rectangular pads made of synthetic or natural rubber or any suitable material.

Referring to FIG. 2, the enclosure 100 includes a power socket 140 or the like, and a power switch 150. In one embodiment, the socket 140 and switch 150 are located on a side wall 110 generally away from the handle 125, i.e., on a side or rear panel side wall of the enclosure 100.

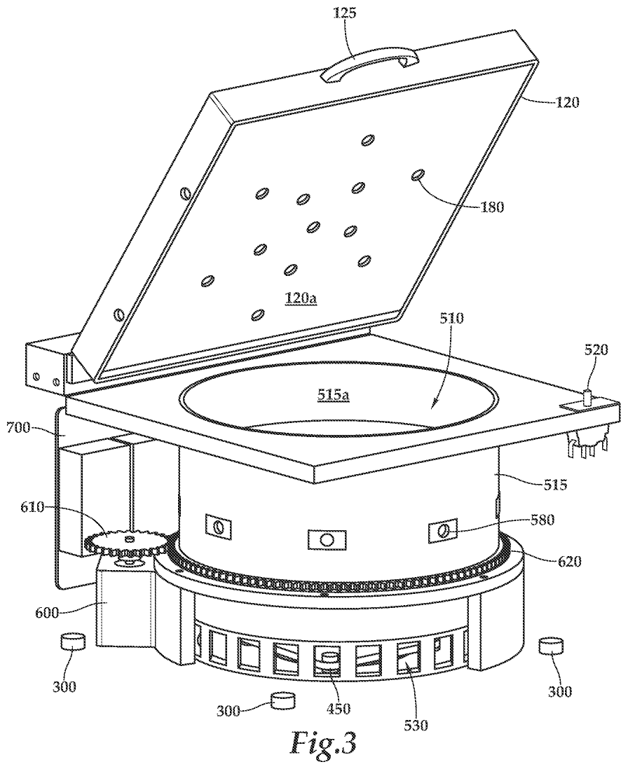

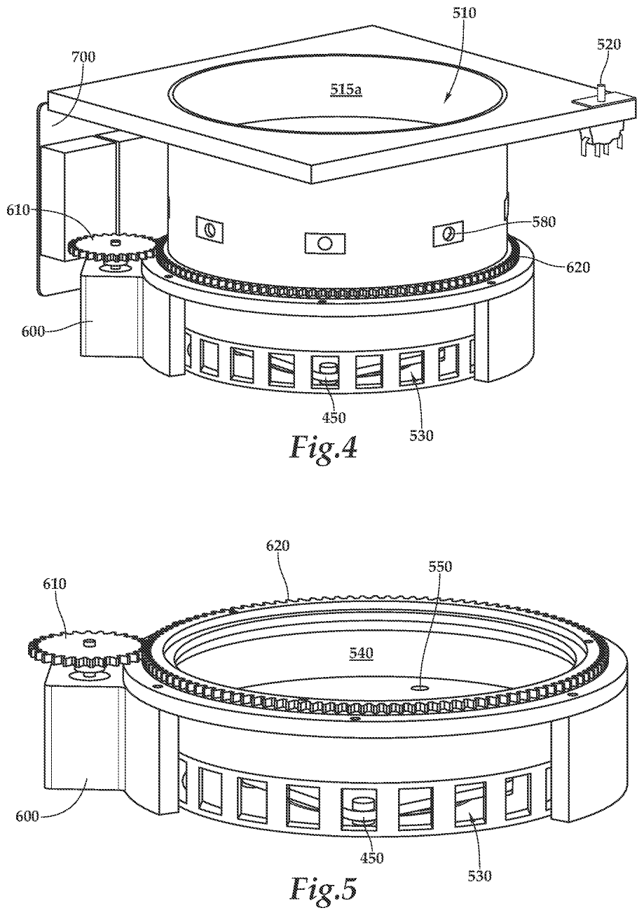

FIGS. 3 and 4 are perspective views of the device 10 with the enclosure 100 removed and the lid 120 partially opened to show some of the elements within the enclosure. FIG. 4 further omits the enclosure 100 and lid 120.

The lid 120 has an interior surface 120a, in which a plurality of UVC light radiation sources 180 are disposed so as to emit UVC radiation into the chamber 510 of the device 10. The UVC light radiation sources 180 may be evenly distributed across the surface 120a or according to any suitable pattern to achieve a selected coverage from the direction of the lid 120 of an object within the chamber 510. For purposes of the present disclosure, the source of UVC radiation may be generated by one or both of UV discharge lamps or UVC LED, for example, with LEDs being preferred. Each UVC LED lamp may be a 50-70 mW UVC LED lamp. The interior lid surface 120a may include about 3 to about 18 UVC light radiation sources 180, for example about 3 sources, about 4 sources, about 5 sources, about 6 sources, about 7 sources, about 8 sources, about 9 sources, about 10 sources, about 11 sources, about 12 sources, about 13 sources, about 14 sources, about 15 sources, about 16 sources, about 17 sources, or about 18 UVC light radiation sources 180.

Each UVC light radiation source 180 emits a cone-shaped pattern 185 of UVC light radiation with a cone angle α of about 75° to about 105°, for example about 75°, about 80°, about 85°, about 90°, about 95°, about 100°, or about 105°. In some embodiments, the UVC light radiation source 180 emits a cone-shaped pattern 185 of UVC light radiation with a cone angle α of about 90°.

The chamber 510 may be a cylindrical space defined, at least in part, by a curving sidewall 515a. Other chamber shapes are contemplated, as long as the shape of such a chamber permits placement of radiation sources in a pattern that enables sufficient radiation coverage of the interior space and object being irradiated.

A plurality of UVC light radiation sources 580 are disposed with the sidewall 515a and configured to emit radiation into the chamber 510. The sidewall 515a may be flange-shaped at an upper end there and provided with a safety cutoff switch 520, such as a plunger-type switch, that is configured to shut off all of the radiation-emitting devices 180, 580, and possibly other aspects of the device 10, when the lid 120 is lifted. Other features of the device 10 can be switched off by operation of the safety cutoff switch 520 by opening the lid 120. The sidewall 515a is fixed within the enclosure 100 such that it is fixed in position. The sidewall 515a may include about 3 to about 18 UVC light radiation sources 580, for example about 3 sources, about 4 sources, about 5 sources, about 6 sources, about 7 sources, about 8 sources, about 9 sources, about 10 sources, about 11 sources, about 12 sources, about 13 sources, about 14 sources, about 15 sources, about 16 sources, about 17 sources, or about 18 UVC light radiation sources 580.

Each UVC light radiation source 580 emits a cone-shaped pattern 585 of UVC light radiation with a cone angle α of about 75° to about 105°, for example about 75°, about 80°, about 85°, about 90°, about 95°, about 100°, or about 105°. In some embodiments, the UVC light radiation source 580 emits a cone-shaped pattern 585 of UVC light radiation with a cone angle α of about 90°.

The sidewall 515a and lid interior surface 120a is made of, coated with, or lined with a highly UV reflective material in order to maximize the distribution of UVC light radiation in the chamber 510. In particular, the material forming the sidewall 515a and interior surface 120a may be a type of polytetrafluoroethylene (PTFE), such as sintered PTFE. Preferably, the PTFE has a reflectivity rate of UVC radiation of greater than about 90%. Preferably, the PTFE has a reflectivity rate of UVC radiation of greater than about 90% of UVC radiation having a wavelength of 265 nm. More preferably, the PTFE has a reflectivity rate of UVC radiation of greater than about 94%. An example of such a PTFE material is “PRM10” by Porex Corporation; Fairburn, Georgia. Even more preferably, the PTFE has a reflectivity rate of UVC radiation of greater than about 97%. An example of such a PTFE material is “PRM15” by Porex Corporation; Fairburn, Georgia. It has been found that PTFE, such as sintered PTFE, can be more effective at reflecting and distributing a greater amount of UVC radiation in a more useful and broad pattern and at a greater range of angles relative to other types of surfaces, such as for example aluminum.

It has been found that incorporating a highly reflective PTFE material, such as a sintered PTFE material, on the lid inner surface 120a and on the sidewalls 515a substantially increases the intensity of the UVC light radiation within the chamber 510. In some embodiments, UVC light radiation within the chamber 510 is at least about 150% more intense, such as about 150% more intense, about 175% more intense, about 200% more intense, about 225% more intense, about 250% more intense, about 275% more intense, about 300% more intense, about 325% more intense, about 350% more intense, about 375% more intense, or about 400% more intense, when the lid inner surface 120a and the sidewalls 515a consist of a highly reflective PTFE material, such as a sintered PTFE material, compared to the same dimensioned chamber 510 wherein the lid inner surface 120a and the sidewalls 515a do not include a highly reflective PTFE material, such as a sintered PTFE material.

The sidewall 515a is positioned over a base gear 620, which in some embodiments is a ring-shaped and hollow construction (see e.g., FIG. 5). The base gear 620 is in operational engagement with a drive gear 610 that is itself driven by a drive motor 600. When the drive motor 600 is activated, the drive gear 610 is caused to rotate and, as a result, the base gear 620 is rotated. Below the base gear 620 is a plurality of air vents 530 that open to a space 460 below the base gear 620. In the space 460 below the base gear 620 is a fan 450 that vents air through the vents 530 into the space 460 below the base gear 620 and cools components of the device 10 during operation. Outside of the sidewall 515a and within the enclosure 100 is a logic board 700 that is configured to operate elements of the device 10. The logic board 700 is in operational communication with one or more of the UVC radiation-emitting devices 180, 580, 550; the safety cutoff switch 520; the fans 400, 450; the control input device 200; the drive motor 600; the power switch 150; and optionally other aspects of the device 10.

The base gear 620, turning to FIG. 5, has disposed within a chamber base 540 that rotates with the base gear 620. The chamber base 540 is made of a material that is transparent or substantially transparent to (e.g., permits the passage of all or substantially all) UVC radiation. One example of a suitable material for the chamber base 540 is quartz glass. Underneath the chamber base 540 is a further source 550 of UVC radiation which directs UVC radiation through the material of the base and into the chamber 510 (FIG. 3). The device 10 may include about 3 to about 18 UVC light radiation base sources 550, for example about 3 base sources, about 4 base sources, about 5 base sources, about 6 base sources, about 7 base sources, about 8 base sources, about 9 base sources, about 10 base sources, about 11 base sources, about 12 base sources, about 13 base sources, about 14 base sources, about 15 base sources, about 16 base sources, about 17 base sources, or about 18 UVC light radiation base sources 180.

Each UVC light radiation source 550 emits a cone-shaped pattern 555 of UVC light radiation with a cone angle α of about 75° to about 105°, for example about 75°, about 80°, about 85°, about 90°, about 95°, about 100°, or about 105°. In some embodiments, the UVC light radiation source 550 emits a cone-shaped pattern 555 of UVC light radiation with a cone angle α of about 90°.

The motor 600 rotates the base gear 620 at a rate that rotates an object within the chamber on the base 540 several times per minute, for example, about 2 to about 30 times per minute such as about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 times per minute. In some embodiments, the motor 600 rotates the base gear 620 at a rate that rotates an object within the chamber on the base 540 about 4 to about 16 times per minute, for example about 4, about 5, about 6, about 7, about 8, about 9, about 10, about 11, about 12, about 13, about 14, about 15, or about 16 times per minute. In some embodiments, the motor 600 rotates the base gear 620 at a rate that rotates an object within the chamber on the base 540 about 8 to about 12 times per minute, for example about 8, about 9, about 10, about 11, or about 12 times per minute. In some embodiments, the motor 600 rotates the base gear 620 at a rate that rotates an object within the chamber on the base 540 about 10 times per minute.

The device 10 is configured to disinfect (e.g., decontaminate) an object in the chamber 510 in about 30 seconds to about 5 minutes, for example in about 30 seconds, in about 1 minute, in about 1.5 minutes, in about 2 minutes, in about 2.5 minutes, in about 3 minutes, in about 3.5 minutes, in about 4 minutes, in about 4.5 minutes, or in about 5 minutes. In some embodiments, the device 10 is configured to disinfect (e.g., decontaminate) an object in the chamber 510 in about 30 seconds to about 2 minutes, for example in about 30 seconds, in about 1 minute, in about 1.5 minutes, or in about 2 minutes. In some embodiments, the device 10 is configured to disinfect (e.g., decontaminate) an object in the chamber 510 in not more than about 1 minute.

In some embodiments, the volume of the chamber 510 is about 50 cubic inches to about 700 cubic inches, for example about 50 cubic inches, about 100 cubic inches, about 150 cubic inches, about 200 cubic inches, about 250 cubic inches, about 300 cubic inches, about 350 cubic inches, about 400 cubic inches, about 450 cubic inches, about 500 cubic inches, about 550 cubic inches, about 600 cubic inches, about 650 cubic inches, or about 700 cubic inches. In some embodiments, the volume of the chamber 510 is about 150 cubic inches to about 500 cubic inches, for example about 150 cubic inches, about 200 cubic inches, about 250 cubic inches, about 300 cubic inches, about 350 cubic inches, about 400 cubic inches, about 450 cubic inches, or about 500 cubic inches. In some embodiments, the volume of the chamber 510 is about 250 cubic inches to about 400 cubic inches, for example about 250 cubic inches, about 260 cubic inches, about 270 cubic inches, about 280 cubic inches, about 290 cubic inches, about 300 cubic inches, about 310 cubic inches, about 320 cubic inches, about 330 cubic inches, about 340 cubic inches, about 350 cubic inches, about 360 cubic inches, about 370 cubic inches, about 380 cubic inches, about 390 cubic inches, or about 400 cubic inches. In one embodiment, the volume of the chamber 510 is about 315 cubic inches.

The base gear 620 which surrounds and/or supports the base 540, the drive gear 610 and drive motor 600 are shown in operational engagement in FIGS. 5-7. Underneath the base 540 is a portion of the device 10 showing positions of the vents 530 and fan 450.

In other embodiments, the base 540 rotates in response to a belt-drive system comprising a drive motor 600, a drive wheel (not shown) in operative communication with the drive motor 600, and a belt (not shown) disposed about the drive wheel and a base wheel (not shown) associated with the base 540.

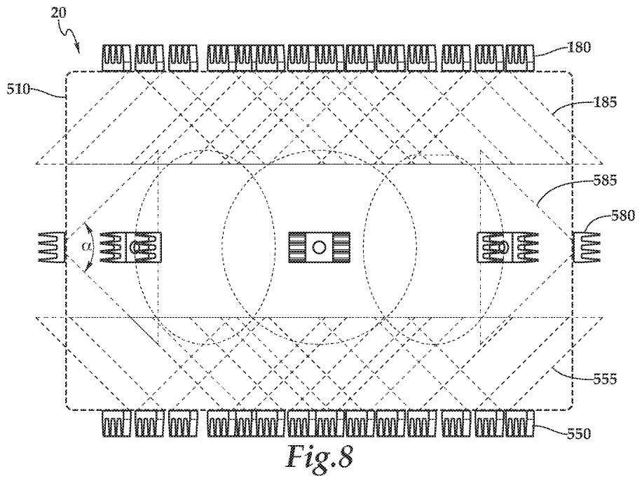

All of the UVC LED sources 180, 550, 580 are positioned emit radiation in an overlapping pattern to eliminate non-irradiated space in the chamber 510, consistent generally with that shown in FIG. 8.

In some embodiments, the device 10 is configured to emit UVC light radiation within the chamber 510 at an average intensity of about 0.5 mW/cm² to about 0.6 mW/cm² at 265 nm. In some embodiments, the intensity of the UVC light radiation is not less than about 0.4 mW/cm² at 265 nm at any point within the chamber 510 (e.g., at any point within the chamber 510 after exactly one rotation of the base 540).

In some embodiments, the device 10 is configured to kill substantially all pathogens on the outer surface of an object placed within the chamber 510. In some embodiments, the device 10 is configured to kill at least 99% (i.e., log-2), at least 99.9% (e.g., log-3), at least 99.99% (e.g., log-4), at least 99.999% (e.g., log-5), or at least 99.9999% (e.g., log-6) of pathogens on the surface of the object in not more than about 2 minutes of exposure to UVC light radiation inside the chamber 510.

In some embodiments, the device 10 is configured to kill at least 99% (i.e., log-2), at least 99.9% (e.g., log-3), at least 99.99% (e.g., log-4), at least 99.999% (e.g., log-5), or at least 99.9999% (e.g., log-6) of pathogens on the surface of the object in not more than about 1 minute of exposure to UVC light radiation inside the chamber 510.

Referring now specifically to FIG. 8, one embodiment of a UVC light radiation pattern generated by the UVC LED sources 180, 550, 580 is shown. For context, the outer dimensions of the chamber 510 are shown in dashed lines. In the illustrated embodiment, each UVC LED source 180, 550, 580 emits a cone-shaped pattern 185, 555, 585 of UVC light radiation with a cone angle α of about 90°. The UVC LED sources 180, 550, 585 are arranged such that their respective cone-shaped patterns 185, 555, 585 of UVC light radiation overlap, as shown by the regions of relatively darker shading in FIG. 8, to reduce or eliminate (e.g., substantially eliminate) areas within the chamber 510 of little or no UVC light radiation. For clarity purposes only, the cone-shaped patterns 185, 555, 585 of UVC light radiation are illustrated having a finite distance of substantially uniform light radiation density. In actuality, the cone-shaped pattern 185, 555, 585 of UVC light radiation emitted by each UVC LED source 180, 550, 580 will be most intense closest to the UVC LED source (i.e., at the apex of the cone-shaped pattern 185, 555, 585), and will decay in intensity ad infinitum consistent with the inverse squared law.

Devices 10 consistent with the present disclosure generally eliminate the need for a rack or other scaffolding within the chamber 510 to support the object during processing. Devices currently available commercially often include metal or plastic racks, hangers, or scaffolding to suspend or support the object, but the racks, hangers, and scaffolding are themselves opaque to UVC light radiation and therefore create shadows on the object that prevent thorough disinfection (e.g., decontamination). Thus, in some embodiments, the device 10 does not include a UVC-opaque support within the chamber 510.

In some embodiments, the present disclosure provides a device 10 for disinfecting an object comprising a surface, the device comprising: a lid 120 including at least one UVC light radiation source 180; a chamber 510 configured to house the object, the chamber comprising: at least one side wall 515b including at least one UVC light radiation source 580, and a rotatable base 540 comprising quartz glass; and at least one UVC light radiation source 550 disposed below the rotatable base. In some embodiments, the at least one side wall 515b includes an inner surface 515a comprising polytetrafluoroethylene (“PTFE”). In some embodiments, the PTFE surface reflects at least about 90% of light radiation having a wavelength of 265 nm. In some embodiments, the lid 120 comprises an inner surface 120a comprising PTFE. In some embodiments, the PTFE surface reflects at least about 90% of light radiation having a wavelength of 265 nm. In some embodiments, the rotatable base 540 is configured to rotate relative to the UVC light radiation sources 550. In some embodiments, the device further comprises a fan 450 configured to force air around but not into the chamber 510. In some embodiments, the UVC light radiation sources 180, 580, 550 are UVC LED lamps. In some embodiments, each UVC LED lamp is a 50-70 mW UVC LED lamp. In some embodiments, each UVC LED lamp emits UVC light radiation in a conical shape having a cone angle α of about 90°. In some embodiments, the rotatable base 540 is configured to rotate at a rate of about 5 rpm to about 20 revolutions per minute (“rpm”). In some embodiments, the rotatable base 540 is configured to rotate at a rate of about 10 rpm. In some embodiments, the device 10 is configured to kill at least 99.9% (e.g., log-3), at least 99.99% (e.g., log-4), at least 99.999% (e.g., log-5), or at least 99.9999% (e.g., log-6) of pathogens on the surface of the object. In some embodiments, the pathogens are killed within about 5 minutes of an initial contact with UVC light radiation from the UVC light radiation sources. In some embodiments, the pathogens are killed within about 3 minutes of an initial contact with UVC light radiation from the UVC light radiation sources. In some embodiments, the pathogens are killed within about 1 minute of an initial contact with UVC light radiation from the UVC light radiation sources.

In some embodiments, the present disclosure provides a chamber 510 comprising UVC light radiation at an average intensity of about 0.5 mW/cm² to about 0.6 mW/cm² at 265 nm. In some embodiments, the intensity of the UVC light radiation is not less than about 0.4 mW/cm² at 265 nm at any point within the chamber. In some embodiments, the chamber is at least partially enclosed by a polytetrafluoroethylene (“PTFE”) surface. In some embodiments, the chamber includes a base 540 comprising, consisting essentially of, or consisting of quartz glass. In some embodiments, the chamber 510 is enclosed by an enclosure 120a, 515a, 540 consisting essentially of PTFE and quartz glass. In some embodiments, the UVC light radiation is supplied by one or more UVC LED lamps 180, 580, 550. In some embodiments, the base 540 rotates relative to a source 550 of the UVC light radiation. In some embodiments, the chamber 510 has a volume of about 200 in³ (about 3.28 L) to about 500 in³ (about 8.19 L). In some embodiments, the chamber 510 has a volume of about 314 in³ (about 5.14 L). In some embodiments, the UVC light radiation is supplied by about 25 to about 40 UVC LED lamps 180, 580, 550. In some embodiments, the UVC light radiation is supplied by not more than 32 UVC LED lamps 180, 580, 550. In some embodiments, a plurality of the UVC LED lamps 180 are disposed on a lid 120 of the chamber 510; a plurality of the UVC LED lamps 550 are disposed below a base 540 of the chamber 510; and a plurality of the UVC LED lamps 580 are disposed about one or more side walls 515 of the chamber 510.

### Example

In an illustrative example, the device 10 includes 12 UVC LED sources 180 in the lid 120a, 8 UVC LED sources 580 distributed in the chamber 510, and 12 UVC LED sources 550 disposed below and directed through the base 540. Each UVC LED light source is a 70 mW UVC LED lamp emitting UVC light radiation in a cone-shaped pattern having a cone angle α of about 90°. The base 540 consists essentially of quartz glass is configured to rotate, via the motor 600, at a rate of about 10 rotations per minute. The lid inner surface 120a and the sidewall 515a consist of a sintered PTFE material configured to reflect about 97% of UVC light radiation (PRM15, Porex Corporation). The chamber 510 is cylindrical in shape and includes a base diameter of about 10 inches and a height of about 4 inches (total volume about 314 cubic inches). This embodiment will disinfect (e.g., decontaminate) an object placed within the chamber 510 to a log-2 or log-3 level (i.e., killing >99% or >99.9%, respectively, of all pathogens on the object's surface) in about 1 minute or less.

### CONCLUSION

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

1-29. (canceled)

30. An enclosure comprising UVC light radiation at an average intensity of about 0.5 mW/cm² to about 0.6 mW/cm² at 265 nm.

31. The enclosure of claim 30, wherein the intensity of the UVC light radiation is not less than about 0.4 mW/cm² at 265 nm at any point within the enclosure.

32. The enclosure of claim 30, wherein the enclosure is at least partially enclosed by a polytetrafluoroethylene (“PTFE”) surface.

33. The enclosure of claim 30, wherein the enclosure includes a base comprising, consisting essentially of, or consisting of quartz glass.

34. The enclosure of claim 30, wherein the enclosure is enclosed by an enclosure consisting essentially of PTFE and quartz glass.

35. The enclosure of claim 33, wherein the UVC light radiation is supplied by one or more UVC LED lamps.

36. The enclosure of claim 30, wherein the base rotates relative to a source of the UVC light radiation.

37. The enclosure of claim 30, wherein the enclosure has a volume of about 200 in³ (about 3.28 L) to about 500 in³ (about 8.19 L).

38. The enclosure of claim 37, wherein the enclosure has a volume of about 314 in³ (about 5.14 L).

39. The enclosure of claim 37, wherein the UVC light radiation is supplied by about 25 to about 40 UVC LED lamps.

40. The enclosure of claim 38, wherein the UVC light radiation is supplied by not more than 32 UVC LED lamps.

41. The enclosure of claim 38, wherein:
a plurality of the UVC LED lamps are disposed on a lid of the chamber;
a plurality of the UVC LED lamps are disposed below a base of the chamber; and
a plurality of the UVC LED lamps are disposed about one or more sidewalls of the chamber.

42. An enclosure comprising:
a rotatable base configured to support an object to be disinfected, the rotatable base configured to rotate the object within the enclosure;
at least one inner sidewall disposed adjacent to the rotatable base; and
a plurality of UVC light radiation sources configured to emit UVC light radiation into the enclosure at an intensity not less than about 0.4 mW/cm² at 265 nm at any point within the enclosure.

43. The enclosure of claim 42, wherein the rotatable base is substantially transparent to UVC radiation.

44. The enclosure of claim 43, wherein at least one of the plurality of UVC light radiation sources is disposed opposite the rotatable base from the object.

45. The enclosure of claim 42, wherein the enclosure does not include a rack or scaffold configured to support the object.

46. The enclosure of claim 42, wherein the inner sidewall forms a cylindrical shape.

47. The enclosure of claim 42 further comprising a lid configured to seal the enclosure sufficient to prevent air and light from exiting the enclosure.

48. The enclosure of claim 47, wherein at least one of the plurality of UVC light radiation sources is disposed in the lid.

49. A device for disinfecting an object, the device comprising:
a rotatable base configured to support an object to be disinfected, the rotatable base configured to rotate the object within the enclosure and to permit substantially all UVC radiation to pass therethrough;
at least one inner sidewall disposed adjacent to the rotatable base;
a lid configured to seal the device sufficient to prevent air and light from exiting the device; and
a plurality of UVC light radiation sources configured to emit UVC light radiation into the enclosure at an intensity not less than about 0.4 mW/cm² at 265 nm at any point within the enclosure,
wherein at least one of the plurality of UVC light radiation sources is disposed opposite the rotatable base from the object,
wherein at least one of the plurality of UVC light radiation sources is disposed in the at least one inner sidewall, and
wherein at least one of the plurality of UVC light radiation sources is disposed in the lid.
